(19) United States
(12) Patent Application Publication
(10) Pub. No.: US 2015/0051614 A1
(43) Pub. Date: Feb. 19, 2015

(54) LEADLESS CARDIAC PACING DEVICES

(71) Applicant: Cardiac Pacemakers, Inc., St. Paul, MN (US)

(72) Inventors: Brian L. Schmidt, White Bear Lake, MN (US); Dana Sachs, Pine City, MN (US); Benjamin J. Haasl, Forest Lake, MN (US); Arthur J. Foster, Blaine, MN (US); Thomas J. Herbst, Coon Rapids, MN (US)

(21) Appl. No.: 14/452,641

(22) Filed: Aug. 6, 2014

Related U.S. Application Data
(60) Provisional application No. 61/866,820, filed on Aug. 16, 2013.

(51) Int. Cl. A61N 1/05; A61N 1/375

(52) U.S. Cl. CPC A61N 1/0573; A61N 1/3756; A61N 2001/058
USPC 606/129; 607/127

(57) ABSTRACT

Implantable leadless pacing devices and medical device systems including an implantable leadless pacing device are disclosed. An example implantable leadless pacing device may include a pacing capsule. The pacing capsule may include a housing. The housing may have a proximal region and a distal region. A first electrode may be disposed along the distal region. An anchoring member may be coupled to the distal region. One or more anti-rotation members may be fixedly attached to the distal region.

### CROSS-REFERENCES TO RELATED APPLICATIONS

This application claims the benefit of U.S. Provisional Application No. 61/866,820 filed Aug. 16, 2013, the complete disclosure of which is herein incorporated by reference.

### TECHNICAL FIELD

The present disclosure pertains to medical devices, and methods for manufacturing medical devices. More particularly, the present disclosure pertains to leadless cardiac pacing devices.

### BACKGROUND

A wide variety of medical devices have been developed for medical use, for example, cardiac use. Some of these devices include catheters, leads, pacemakers, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example implantable leadless pacing device may include a pacing capsule. The pacing capsule may include a housing. The housing may have a proximal region and a distal region. A first electrode may be disposed along the distal region. An anchoring member may be coupled to the distal region. One or more anti-rotation members may be coupled to the distal region. The anti-rotation members may be capable of breaking when exposed to retrieval forces.

An example implantable leadless pacing device system may include a delivery catheter having a proximal section, a distal holding section, and a lumen formed therein. A push member may be slidably disposed within the lumen. A leadless pacing device may be slidably received within the distal holding section. The leadless pacing device may include a housing having a proximal region and a distal region. A first electrode may be disposed along the distal region. An anchoring member may be coupled to the distal region. One or more anti-rotation members may be coupled to the distal region. The anti-rotation members may be capable of breaking when exposed to retrieval forces.

Another example implantable leadless pacing device system may include a delivery catheter having a proximal section, a distal holding section, and a lumen formed therein. A push member may be slidably disposed within the lumen. A leadless pacing device may be slidably received within the distal holding section. The leadless pacing device may include a housing having a proximal region and a distal region. A first electrode may be disposed along the distal region. A helical anchoring member may be coupled to the distal region. A plurality of anti-rotation members may be fixedly attached to the distal region and spaced from the helical anchoring member. The anti-rotation members may be capable of breaking when exposed to retrieval forces.

Another example implantable leadless pacing device system may include a delivery catheter having a proximal section, a distal holding section, and a lumen formed therein. A push member may be slidably disposed within the lumen. A leadless pacing device may be slidably received within the distal holding section. The leadless pacing device may include a housing having a proximal region and a distal region. A first electrode may be disposed along the distal region. A helical anchoring member may be coupled to the distal region. A plurality of breakable anti-rotation tines may be coupled to the housing.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan view of an example leadless pacing device implanted within a heart;

FIG. 2 is a side view of an example leadless pacing device;

FIG. 2A is a distal end view of the example leadless pacing device shown in FIG. 2;

FIG. 2B is a proximal end view of the example leadless pacing device shown in FIG. 2;

FIG. 2C is a plan view of the example leadless pacing device shown in FIG. 2 implanted within a cardiac tissue;

FIG. 3 is a plan view of an example leadless pacing device being retrieved from the cardiac tissue; and

FIGS. 4A-4F are side views of example anti-rotation members. is a side view of another example leadless pacing device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Cardiac pacemakers provide electrical stimulation to heart tissue to cause the heart to contract and thus pump blood through the vascular system. Conventional pacemakers typically include an electrical lead that extends from a pulse generator implanted subcutaneously or sub-muscularly to an electrode positioned adjacent the inside or outside wall of the cardiac chamber. As an alternative to conventional pacemakers, self-contained or leadless cardiac pacemakers have been proposed. A leadless cardiac pacemaker may take the form of a relatively small capsule that may be fixed to an intracardiac implant site in a cardiac chamber. It can be readily appreciated that the implantation of a leadless pacing device within a beating heart could become dislodged as the heart functions. Accordingly, it may be desirable for a leadless pacing device to include an anchoring mechanism and/or one or more anchoring members to help securing the pacing device to the heart.

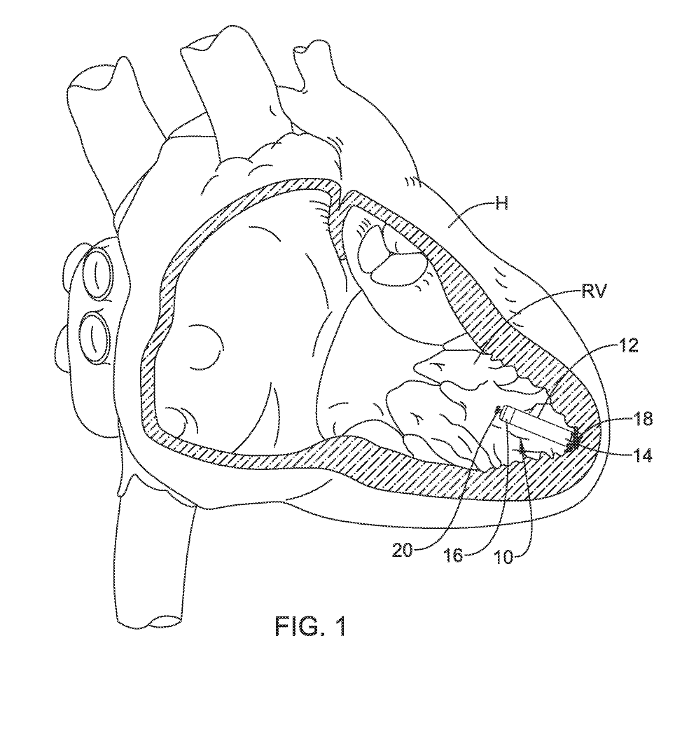

FIG. 1 illustrates an example implantable leadless cardiac pacing device 10 implanted in a chamber of a heart H such as, for example, the right ventricle RV. Device 10 may include a shell or housing 12 having a distal region 14 and a proximal region 16. One or more anchoring members 18 may be disposed adjacent to distal region 14. Anchoring member 18 may be used to attach device 10 to a tissue wall of the heart H, or otherwise anchor implantable device 10 to the anatomy of the patient. A docking member 20 may be disposed adjacent to proximal region 16 of housing 12. Docking member 20 may be utilized to facilitate delivery and/or retrieval of implantable device 10.

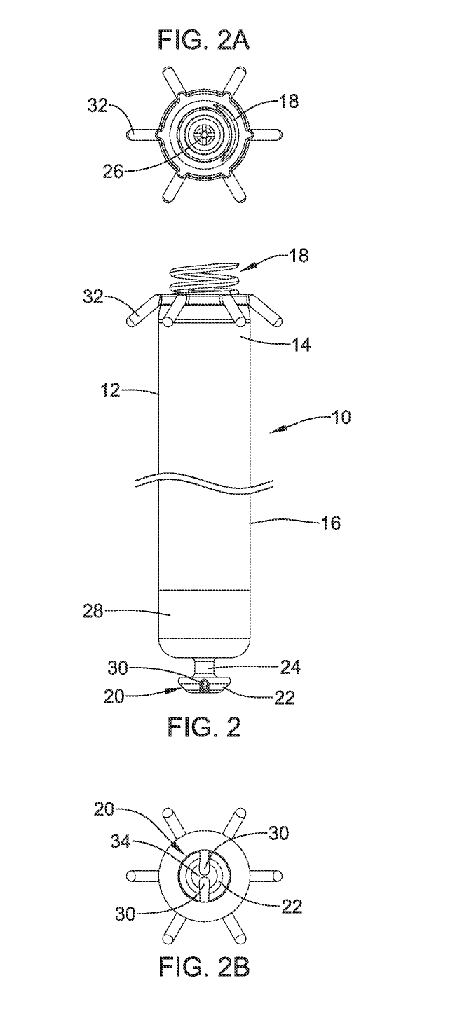

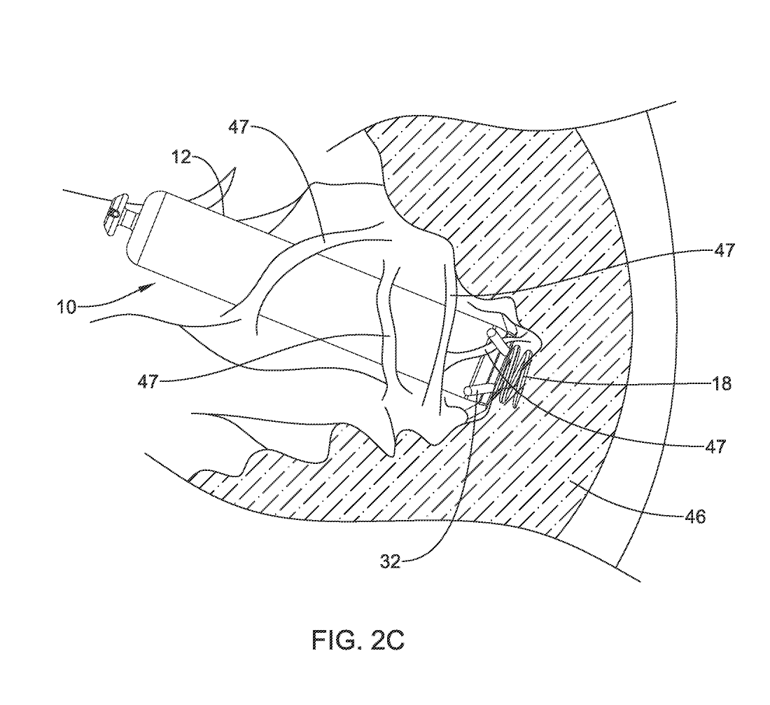

Some of the features of device 10 can be seen in FIG. 2, FIG. 2A, and FIG. 2B. For example, device 10 may include a first electrode 26 positioned adjacent to the distal region 14 of the housing 12. A second electrode 28 may also be defined along housing 12. For example, housing 12 may include a conductive material and may be insulated along a portion of its length. A section along proximal region 16 may be free of insulation so as to define second electrode 28. Electrodes 26/28 may be sensing and/or pacing electrodes to provide electro-therapy and/or sensing capabilities. First electrode 26 may be capable of being positioned against or otherwise contact the cardiac tissue of the heart H while second electrode 28 may be spaced away from the first electrode 26, and thus spaced away from the cardiac tissue. Device 10 may also include a pulse generator (e.g., electrical circuitry) and a power source (e.g., a battery) within housing 12 to provide electrical signals to electrodes 26/28. Electrical communication between pulse generator and electrodes 26/28 may provide electrical stimulation to heart tissue and/or sense a physiological condition.

Docking member 20 may include a head portion 22 and a neck portion 24 extending between housing 12 and head portion 22. Head portion 22 may be capable of engaging with a delivery and/or retrieval catheter. For example, head portion 22 may include a bore or opening 30 formed therein. The ends of bore 30 may be open or exposed while a central region of bore 30 may be covered by a section 34 of head portion. During delivery, device 10 may be secured to a delivery device by extending a suture through bore 30. A portion of the delivery catheter may include projections or lugs that may engage bore 30. These are just examples. A variety of delivery devices are contemplated.

Docking member 20 may also be engaged if it is desired to retrieve and/or reposition device 10. For example, a retrieval catheter may be advanced to a position adjacent to device 10. A retrieval mechanism such as a snare, tether, arm, or other suitable structure may extend from the retrieval catheter and engage head portion 22. When suitably engaged, device 10 may be pulled from the cardiac tissue and, ultimately, removed from the patient or repositioned.

As the name suggest, anchoring member 18 may be used to anchor device 10 to the target tissue. A suitable number of anchoring member 18 may be used with device 10. For example, device 10 may include one, two, three, four, five, six, seven, eight, or more anchoring members. In at least some embodiments, anchoring member 18 may take the form of a helix or screw. According to these embodiments, anchoring member 18 may be threaded into cardiac tissue. For example, a portion of a delivery catheter (e.g., a push member) may be capable of engaging docking member such that rotation of the push member may cause anchoring member 18 to thread into cardiac tissue.

It can be appreciated that in order to securely anchor device 10 to cardiac tissue with a helical anchoring member 18, it may be desirable to reduce or prevent unintended rotation and/or “unthreading” of device 10. Because of this device, device 10 may include one or more anti-rotation tines or members 32. In general, anti-rotation members 32 may be disposed along distal region 14 and may extend radially outward from housing 12. In at least some embodiments, anti-rotation members 32 may help to maintain device 10 in a securely anchored arrangement. For example, FIG. 2C illustrates device 10 implanted within a cardiac tissue 46. In this example, cardiac tissue 46 may have a number of trabeculae 47 along the surface thereof. Anti-rotation members 32 may become entwined with trabeculae 47 so that unwanted rotation of device 10 may be reduced and/or prevented.

Anti-rotation members 32 may be fixedly attached to housing 12. In other words, anti-rotation members may be designed so that during typical use, anti-rotation members 32 remain attached to housing 12. In some embodiments, anti-rotation member 32 may have some freedom of movement relative to housing 12. For example, anti-rotation members 32 may be capable of pivoting, rotating, or otherwise moving relative to housing 12. The form of anti-rotation members 32 may vary. For example, anti-rotation members 32 may take the form of cylindrical rods or tubes projecting from housing 12. The rods may have a generally circular cross-sectional shape. In at least some embodiments, anti-rotation members 32 may be substantially straight. In other embodiments, anti-rotation members 32 may include one or more curves or bends. A variety of other shapes, forms, and configurations are also contemplated for anti-rotation members 32 and some of these are disclosed herein. In addition, some devices may include combinations of differently shaped or oriented anti-rotation members 32.

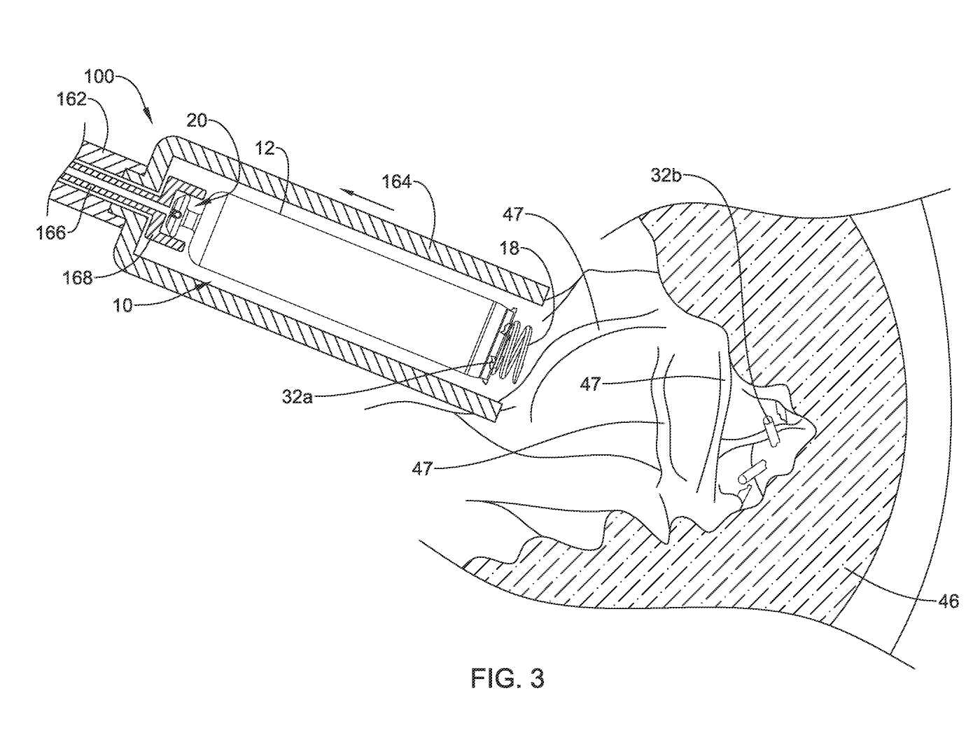

At some point, it may become desirable to retrieve and/or reposition device 10. To do so, a retrieval catheter 100 may be used to engage device 10 as depicted in FIG. 3. Catheter 100 may include a proximal member or region 162 and a distal member or holding section 164. A push member 166 may be disposed (e.g., slidably disposed) within proximal region 162. A distal or head region 168 of push member 166 may be disposed within distal holding section 164. Head region 168 may be capable of engaging docking member 20 of device 10 so that device 10 may be retrieved and/or repositioned. Other devices and/or portions of catheter 100 may also be used to engage docking member 20.

Because anti-rotation members 32 may be engaged/entwined with trabeculae 47 and/or may be encapsulated or otherwise engaged with scar tissue that may be formed in the heart adjacent to device 10 (e.g., scar tissue that may be present adjacent to the implant site of device 10), anti-rotation members 32 could provide resistance to the retrieval of device 10. In order to facilitate retrieval of device 10, at least a portion of anti-rotation members 32 may be capable of breaking of or otherwise severing from device 10 when device 10 is exposed to retrieval forces (e.g., forces applied to device 10 during the retrieval thereof from a patient). In doing so, a portion 32a of anti-rotation members 32 may remain coupled to device 10 and another portion 32b may break away from portion 32a and be left behind, entwined with trabeculae 47 and/or encapsulated by scar tissue. In at least some embodiments, portions (e.g., portion 32b) or all of anti-rotation members 32 may be formed from a bio-absorbable or dissolvable material so that portions 32b may be absorbed or dissolved within the body after breaking

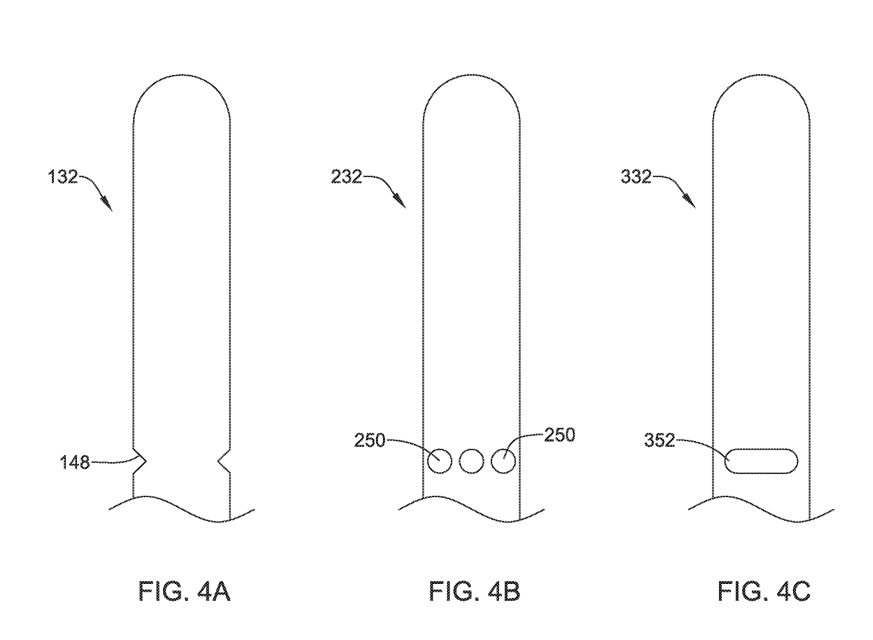

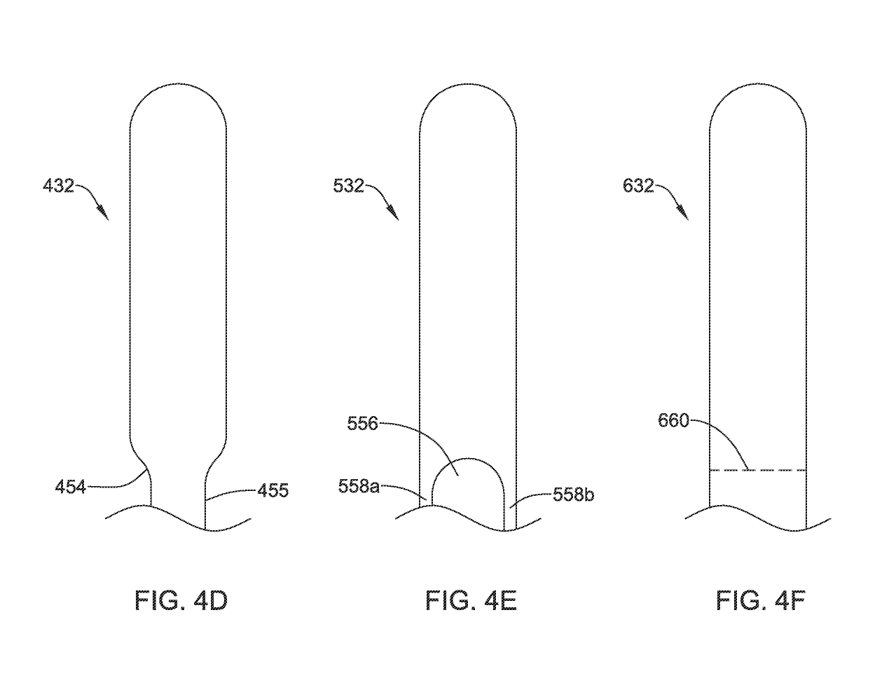

The manner or mechanism for breaking anti-rotation members 32 may vary. For example, anti-rotation members 32 may be formed from a generally breakable or fracturable material so that sufficient force being applied to anti-rotation members 32 (e.g., during retrieval of device 10) may cause breakage. Other forms and/or configurations are contemplated. FIGS. 4A-4F illustrate some of the alternative anti-rotation members contemplated. For example, FIG. 4A illustrates anti-rotation member 132 with a notch or groove 148 formed therein. Notch 148 may define a location along anti-rotation member 132 where breakage may occur. FIG. 4B illustrates anti-rotation member 232 with a plurality of openings 250 formed therein. Openings 250 may define a location along anti-rotation member 232 where breakage may occur. FIG. 4C illustrates anti-rotation member 332 with a relatively wide opening 352 formed therein. Opening 352 may define a location along anti-rotation member 332 where breakage may occur. FIG. 4D illustrates anti-rotation member 432 with a tapered region 454 and a narrowed region 455. Region 454 may define a location along anti-rotation member 432 where breakage may occur. FIG. 4E illustrates anti-rotation member 532 with an opening 556 formed therein that may define thinned segments 558a/558b. Segments 558a/558b may define a location along anti-rotation member 532 where breakage may occur. FIG. 4F illustrates anti-rotation member 632 with a perforation 660 formed therein. Perforation 660 may define a location along anti-rotation member 632 where breakage may occur. These are just examples. Other shapes, forms, and configurations are contemplated.

The materials that can be used for the various components of device 10 and catheter 100 (and/or other devices/catheters disclosed herein) may include those commonly associated with medical devices. For example, device 10 and/or catheter 100 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of device 10 and/or catheter 100 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 and/or catheter 100 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 10 and/or catheter 100 to achieve the same result.

In some embodiments, a degree of Magnetic Resonance Imaging (MRI) compatibility is imparted into device 10 and/or catheter 100. For example, device 10 and/or catheter 100 (or portions thereof) may be made of a material that does not substantially distort the image and create substantial artifacts (i.e., gaps in the image). Certain ferromagnetic materials, for example, may not be suitable because they may create artifacts in an MRI image. Device 10 and/or catheter 100 (or portions thereof) may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable leadless pacing device, comprising:
a pacing capsule including a housing, the housing having a proximal region and a distal region;
a first electrode disposed along the distal region;
an anchoring member coupled to the distal region; and
one or more anti-rotation members coupled to the distal region, the anti-rotation members being capable of breaking when exposed to retrieval forces.

2. The implantable leadless pacing device of claim 1, wherein each of the one or more anti-rotation members includes a region for preferential breaking the one or more anti-rotation members.

3. The implantable leadless pacing device of claim 1, wherein at least some of the anti-rotation members include a bio-absorbable material.

4. The implantable leadless pacing device of claim 1, wherein at least some of the anti-rotation members have a notch formed therein.

5. The implantable leadless pacing device of claim 1, wherein at least some of the anti-rotation members have one or more openings formed therein.

6. The implantable leadless pacing device of claim 1, wherein at least some of the anti-rotation members have a narrowed region.

7. The implantable leadless pacing device of claim 1, wherein at least some of the anti-rotation members have a perforation formed therein.

8. An implantable leadless pacing device system, the system comprising:
a delivery catheter having a proximal section, a distal holding section, and a lumen formed therein;
a push member slidably disposed within the lumen;
a leadless pacing device slidably received within the distal holding section, the leadless pacing device comprising:
a housing having a proximal region and a distal region,
a first electrode disposed along the distal region,
an anchoring member coupled to the distal region, and
one or more anti-rotation members coupled to the distal region, the anti-rotation members being capable of breaking when exposed to retrieval forces.

9. The system of claim 8, wherein each of the one or more anti-rotation members includes a region for preferential breaking the one or more anti-rotation members.

10. The system of claim 8, wherein at least some of the anti-rotation members include a bio-absorbable material.

11. The system of claim 8, wherein at least some of the anti-rotation members have a notch formed therein.

12. The system of claim 8, wherein at least some of the anti-rotation members have one or more openings formed therein.

13. The system of claim 8, wherein at least some of the anti-rotation members have a narrowed region.

14. The system of claim 8, wherein at least some of the anti-rotation members have a perforation formed therein.

15. An implantable leadless pacing device system, the system comprising:
a delivery catheter having a proximal section, a distal holding section, and a lumen formed therein;
a push member slidably disposed within the lumen;
a leadless pacing device slidably received within the distal holding section, the leadless pacing device comprising:
a housing having a proximal region and a distal region,
a first electrode disposed along the distal region,
a helical anchoring member coupled to the distal region, and
a plurality of anti-rotation members fixedly attached to the distal region and spaced from the helical anchoring member, the anti-rotation members being capable of breaking when exposed to retrieval forces.

16. The system of claim 15, wherein each of the one or more anti-rotation members includes a region for preferential breaking the one or more anti-rotation members.

17. The system of claim 16, wherein the region for preferential breaking includes a notch formed therein.

18. The system of claim 16, wherein the region for preferential breaking includes one or more openings formed therein.

19. The system of claim 16, wherein the region for preferential breaking includes a narrowed region.

20. The system of claim 16, wherein the region for preferential breaking includes a perforation formed therein.
